(19) United States
(12) Patent Application Publication
(10) Pub. No.: US 2012/0178904 A1
(43) Pub. Date: Jul. 12, 2012

(54) PEPTIDE ANALOGS THAT ARE POTENT AND SELECTIVE FOR HUMAN NEUROTENSIN RECEPTOR SUBTYPE 2

(76) Inventors: Elliot RICHELSON, Ponte Vedra, FL (US); Daniel J. McCORMICK, Rochester, MN (US); Yuan-Ping PANG, Rochester, MN (US); Kenneth S. PHILLIPS, Corona, CA (US)

(21) Appl. No.: 13/177,909

(22) Filed: Jul. 7, 2011

Related U.S. Application Data
(62) Division of application No. 11/800,975, filed on May 7, 2007, now abandoned.

(51) Int. Cl. C07K 7/06

(52) U.S. Cl. 530/329; 530/330

(57) ABSTRACT

Neurotensin analogs selective for neurotensin receptor subtype 2 are described. These include hexapeptides (NT(8-13)) and pentapeptides (NT(9-13)) having a D-3,1-naphthyl-alanine, D-3,2-naphthyl-alanine, an alanine derivative such as cyclohexylalanine, or 1,2,3,4-tetrahydroisoquinoline at position 11. Methods of treating pain by administering these neurotensin analogs are also described.

### CROSS-REFERENCES TO RELATED APPLICATIONS

This is a divisional of U.S. Application Serial No. 11/800,975, filed on May 7, 2007, which is hereby expressly incorporated by reference in its entirety.

### BACKGROUND OF THE INVENTION

Polypeptides as well as many other types of compounds such as neurotransmitters and drugs can exert profound effects on the body. For example, neurotensin (NT) induces antinociception and hypothermia upon direct administration to brain. Systemic administration of NT does not induce these effects since NT is rapidly degraded by proteases and has poor blood brain barrier permeability.

Neurotensin is a tridecapeptide with the amino acid sequence pyroGlu-Leu-Tyr-Glu-Asn-Lys-Pro-Arg-Arg-Pro-Tyr-Ile-Leu-OH. Most, if not all, of the activity mediated by NT(1-13) is mediated by the 6 amino acid fragment, NT(8-13), which does not exist naturally in vivo. In order to observe pharmacological effects of either NT or NT(8-13) in the nervous system, each has to be administered directly into the brain or the spinal cord. Intravenous injection of NT and its fragments, however, causes hypotension, as well as other pharmacological effects. (See Carraway, R. et al. J BIOL CHEM 248:6854-61 (1973) and Carraway, R. E. et al. “Structural requirements for the biological activity of neurotensin, a new vasoactive peptide.” In Fourth American Peptide Symposium. Edited by R Walter and J Meienhofer, Ann Arbor Science Publishers Inc., p. 679-85 (1975))

Neurotensin acts as a neurotransmitter or neuromodulator in the central nervous system (CNS), interacting largely with dopaminergic systems. (See Tyler-McMahon, B. M. et al. REGUL PEPT93:125-36 (2000) and Binder, E. B. et al. PHARMACOL REV 53:453-86 (2001)) In addition, it has been known for a long time that neurotensin, when injected into brain, is a potent antinociceptive agent, operating by a p-opioid independent mechanism. (See Clineschmidt, B. V.

Patent et al. EUR J PHARMACOL 46:395-6 (1977) and Clineschmidt, B. V. et al. EUR J PHARMACOL 54:129-39 (1979)) In fact, on a molar basis, NT is more potent than morphine as an antinociceptive agent. (See Nemeroff, C. B. et al. PROC NATL ACAD Sci USA 76:5368-71 (1979) and Al-Rodhan, N. R. et al. BRAIN RESEARCH 557:227-35 (1991))

Neurotensin and its analogs are also potent analgesics in animals. NT is produced in the brain, spinal cord dorsal horn, hypothalamus, and gut. NT receptors involved in the treatment of central pain may be different from those involved in the treatment of peripheral pain. Additionally, NT administration is associated with not just analgesia but also hypotension (unrelated to histamine release), fall in basal temperature, and decreased food intake leading to weight loss. NT has also been known to induce tolerance, increase gastrointestinal transit, induce diarrhea, and exhibit antipsychotic and antiparkinsonian effects (Boules, M. et al., Peptides 27:2523-33 (2006)).

Neurotensin mediates its effects through at least 3 different receptors. (See Boules, M. et al. “NTS1 neurotensin receptor” In xPharm. Edited by S J Enna and D B Bylund. New York City, Elsevier, Inc. (2004); Boules, M. et al. “NTS2 neurotensin receptor” In xPharm. Edited by S J Enna and D B Bylund. New York City, Elsevier, Inc. (2004); and Boules, M. et al. “NTS3 neurotensin receptor” In xPharm. Edited by S J Enna and D B Bylund. New York City, Elsevier, Inc. (2004)) The first neurotensin receptor (NTS1) was molecularly cloned from rat brain (see Tanaka, K. et al. NEURON 4:847-54 (1990)) and human brain (see Watson, M. et al. MAYO CLINIC PROCEEDINGS68:1043-8 (1993)). The second neurotensin receptor (NTS2), which in binding assays is sensitive to the antihistamine levocabastine, has been cloned from mouse (see Mazella, J. et al. J NEUROSCI 16:5613-20 (1996), rat (see Chalon, P. et al. FEBS LETTERS 386:91-4 (1996), and human (see Vita, N. et al. SOCIETY FOR NEUROSCIENCE 23:394 [abstract] (1997)). Both NTS1 and NTS2 are 7-transmembrane spanning, G-protein coupled receptors. A third neurotensin receptor (NTS3) is a transmembrane protein, but spans the membrane only once and is identical to the protein called “sortilin.” (See Mazella, J. et al. J BIOL CHEM 273:26273-6 (1998)). Recent data suggest that NTS3 has a function in inflammatory processes in the central nervous system. (See Martin, S. et al. J NEUROSCI 23:1198-205 (2003)) NT and NT(8-13) have highest affinity for NTS1, followed by NTS2 and NTS3. These peptides have over 1000-fold lower affinity for NTS3, as compared to that for NTS1. (See Kokko, K. P. et al. J MED CHEM 46:4141-8 (2003)). It is likely that both NTS1 and NTS2 mediate the antinociceptive effects of NT (see Dobner, P. R. PEPTIDES27:2405-14 (2006)), while NTS1 mediates the hypotensive effects, among others.

In addition to the antihistamine levocabastine, which has selectivity for NTS2, there are two other non-peptide neurotensin receptor antagonists. One antagonist, SR48692 (see Gully, D. et al. PROC NATL ACAD USA 90:65-9 (1993)), has relatively high affinity for both NTS1 and NTS2, with selectivity for NTS1. (See Chalon, P. et al. FEBS LETTERS386:91-4 (1996)). SR48692 has very low affinity for NTS3. (See Mazella, J. et al. J BIOL CHEM 273:26273-6 (1998)). Consistent with its relative selectivity for NTS1, in vivo SR48692 does not block all the effects of neurotensin. Another antagonist, SR142948A (see Gully, D. et al. J PHARMACOL EXP THER 280:802-12 (1997), has a broader spectrum of activity in vivo against NT and is considered non-selective in binding to NTS1 and NTS2. Its affinity for NTS3 is unknown. Levocabastine may be a partial agonist/antagonist at NTS2. (See Dubuc, I. et al. EUR J PHARMACOL 381:9-12 (1999))

There are many known neurotensin receptor agonists that are non-selective for NTS1 or NTS2 and that are active in the central nervous system (CNS) after peripheral administration (e.g., subcutaneously or intraperitoneally). (See Tyler, B. M. et al. NEUROPHARMACOLOGY 38:1027-34 (1999); Cusack, B. et al. BRAIN RES 856:48-54 (2000); Boules, M. et al. BRAIN RES 919:1-11 (2001); Kokko, K. P. et al. NEUROPHARMACOLOGY 48:417-25 (2005); and Hadden, M. K. et al. NEUROPHARMACOLOGY (2005)). Such results indicate that these non-selective compounds pass the blood-brain barrier (BBB). There are also a few compounds that are relatively selective and potent at rodent NTS2 (e.g., JMV 431) (See Dubuc, I. et al. J NEUROSCI 19:503-10 (1999)) For the published NTS2-selective compounds, however, all studies employed their direct injection into brain (see Dubuc, I. et al. J NEUROSCI 19:503-10 (1999)) or into spinal cord (see Sarret, P. et al. J NEUROSCI 25:8188-96 (2005)) to elicit pharmacological effects. Therefore, it is assumed that these compounds do not penetrate the BBB.

Over the years, Doctor Richelson and his team have designed, synthesized, and tested in vitro and in vivo over 60 peptides that are largely analogs of NT(8-13) and NT(9-13). From these studies, a large amount of structure-activity data were gathered, which led to defining the binding site for NT(8-13) at rat and human NTS1. (See Pang, Y. P. et al. J BIOL CHEM 271:15060-8 (1996)) In addition, brain-penetrating analogs that bind with improved affinity to human NTS1 have been developed, largely as a result of the incorporation into these peptides of a novel amino acid, neo-Trp. (See Fauq, A. H. et al. TETRAHEDRON: ASYMMETRY 9:4127-34 (1998)) This amino acid is a regioisomer of tryptophan. U.S. Patents have been issued for this new amino acid and peptides that contain it, specifically many of the NT agonists developed in the laboratory of Dr. Richelson. (See U.S. Pat. Nos. 6,214,790; 6,765,099; and 7,098,307)

In their series of peptides studied at hNTS1 and hNTS2, about one-half of the compounds had essentially the same affinities for both hNTS1 and hNTS2. Furthermore, there was a strong correlation between the log K_{d} (equilibrium dissociation constant) at hNTS1 and the log K_{d} at hNTS2 for the peptides, indicating that the binding site for these peptides at the hNTS2 is in a region with high homology to the binding site in the hNTS1.

The key binding segment of the NTS1 receptor was previously shown to be the third outer loop of this putative seven-helix transmembrane spanning receptor. (See Pang, Y. P. et al. J BIOL CHEM 271:15060-8 (1996); Cusack, B. et al. J BIOL CHEM 271:15054-9 (1996); and Cusack, B. et al. BIOCHEM PHARMACOL 60:793-801 (2000)) From their computer modeling studies, the binding site for NT(8-13) was determined to be primarily composed of eight residues—Phe³²⁶, Ile³²⁹, Trp³³⁴, Phe³³⁷, Tyr³³⁹, Phe³⁴¹, Tyr³⁴², and Tyr³⁴⁴—in the human NTS1. (See Pang, Y. P. et al. J BIOL CHEM 271:15060-8 (1996)) Seven of the eight hydrophobic residues form an aromatic core of the NT(8-13) binding site or “pocket” in human NTS1.

The human NTS1 (hNTS1) contains 418 amino acids, while hNTS2 is 8 amino acids shorter. Alignment of these receptors shows only about 33% identity of amino acids. The putative third extracellular loop for hNTS1 encompasses amino acids 326-345: FCYISDEQWTPFLYDFYHYF; while the corresponding region for hNTS2 spans amino acids 320-339: YCYVPDDAWTDPLYNFYHYF. In this region, the amino acid identity between the two receptors is still only 60%, but nearly twice as great as the overall figure for these receptors. Of the eight residues of the proposed binding site in hNTS1 (see Pang, Y. P. et al. J BIOL CHEM 271:15060-8 (1996)), five (63%) are identical to those in hNTS2. All the aromatic residues in the third extracellular loop of the two receptors are conserved. In addition, those three residues that are different in the third extracellular loop have almost the same preference for adopting a loop conformation, based upon Chou and Fasman probabilities (see Chou, P. Y. et al. BIOCHEMISTRY 13:211-22 (1974)). From this sequence analogy and from the binding data, the binding site at the hNTS2 is likely composed of eight residues, namely, Tyr³²⁰ Val³²³ Trp³²⁸ Pro³³¹ Tyr³³³ Phe³³⁵ Tyr³³⁶ Tyr³³⁸. The binding pocket of the hNTS2 is just a bit smaller than that of the hNTS1. At the hNTS1, the low affinity of NT50, which is the most selective compound for the hNTS2, is probably due to the steric hindrance introduced most likely by Gln³³³, which is next to the key residue Trp³³⁴ in the hNTS1 and mutated to Ala in hNTS2.

From antisense studies, it appears that the hypothermic effects of neurotensin are mediated by NTS1 in rats and in mice, while antinociceptive effects of NT are mediated by activation of NTS1 in rats and NTS2 in mice. (See Tyler, B. M. et al. PROC NATL ASAD SCI USA 96: 7053-58 (1999) and Dubuc, I. et al. J NEUROSCI 19: 503-10 (1999)).

Curiously, in vitro, antagonists and agonists at the NTS1 have opposite effects at the NTS2. Thus, from studies with the molecularly cloned NTS2, the expected antagonists, SR 48692 and SR 142948A behave as agonists, while NT and other agonists behave as antagonists or partial agonists. (See Vita, N. et al. EUR J PHARMACOL 360: 265-72 (1998) and Yamada, M. et al. LIFE SCI 62: L375-PL380 (1998)). These results are also made more interesting in light of the in vivo studies suggesting that the antagonists SR 48692 and SR 142948A have no intrinsic activities. (See Gully, D. et al. J PHARMACOL EXP THER 280: 802-12 (1997)). Thus, there is a need for selective NTS1 and NTS2 agonists for in vivo experimentation.

Furthermore, NTS2 has been shown to regulate pain. Therefore, we have discovered that compounds selective for NTS2 are effective and selective to treat pain while unexpectedly reducing or eliminating hypotensive effects. Thus, it would be advantageous to discover and develop drugs that selectively regulate NTS2.

### SUMMARY OF THE INVENTION

In one embodiment of the invention, neurotensin analogs that are hexapeptides designated NT(8-13) having a D-3,1-naphthyl-alanine at position 11 are described. Additionally, the neurotensin analog may include an N-methyl-arginine at position 8. Additionally, or in the alternative, the neurotensin analog may include a tert-leucine at position 12. Additionally, or in the alternative, the neurotensin analog may include a diaminobutyric acid at position 9. Additionally, or in the alternative, the neurotensin analog may include a Lysine (D or L) at position 8 or 9. Additionally, or in the alternative, the neurotensin analog may include an Ornithine (D or L) at position 9.

In an alternative embodiment, neurotensin analogs that are pentapeptides designated NT(9-13) having a D-3,1-naphthyl-alanine (D or L) at position 11 are described. Additionally, the neurotensin analog may include a diaminobutyric acid at position 9. In the alternative, the neurotensin analog may additionally include a Lysine (D or L) at position 9. Additionally, or in the alternative, the neurotensin analog may include a tert-leucine at position 12.

In one embodiment of the invention, neurotensin analogs that are hexapeptides designated NT(8-13) having a D-3,2-naphthyl-alanine at position 11 are described, with the proviso that positions 8 and 9 are not Lysine. Additionally, the neurotensin analog may include an N-methyl-arginine at position 8. Additionally, or in the alternative, the neurotensin analog may include a tert-leucine at position 12. Additionally, or in the alternative, the neurotensin analog may include a diaminobutyric acid at position 9. Additionally, or in the alternative, the neurotensin analog may include an Ornithine (D or L) at position 9.

In one embodiment of the invention, neurotensin analogs that are hexapeptides designated NT(8-13) having a D-3,2-naphthyl-alanine at position 11 and an Arginine or an Arginine derivative at position 8 and/or position 9, i.e., at at least one of positions 8 or 9, are described. The Arginine may have an L or D configuration. The Arginine derivative may be N-methyl-arginine. Additionally, or in the alternative, the neurotensin analog may include a diaminobutyric acid at position 9. Additionally, or in the alternative, the neurotensin analog may include a Lysine at position 9. Additionally, or in the alternative, the neurotensin analog may include a tert-leucine at position 12. In one embodiment, the neurotensin analog may have an Arginine at both positions 8 and 9. In another embodiment, the neurotensin analog may have an N-methyl-arginine at position 8. In another embodiment, the hexapeptide has the Arginine or the Arginine derivative at position 8 and an Ornithine at position 9. In another alternative embodiment, the hexapeptide has a Lysine at position 8 and an Arginine at position 9.

In another embodiment, neurotensin analogs that are pentapeptides designated NT(9-13) having a D-3,2-naphthyl-alanine at position 11 are described. The D-3,2-naphthyl-alanine may have a D or L configuration. Additionally, the neurotensin analog may include a tert-leucine at position 12. Additionally, or in the alternative, the neurotensin analog may include a Lysine at position 9. Additionally, or in the alternative, the neurotensin analog may include a diaminobutyric acid at position 9.

In an alternative embodiment, neurotensin analogs that are hexapeptides designated NT(8-13) having an Alanine derivative at position 11 are described. In one embodiment, the Alanine derivative may be cyclohexylalanine.

In an alternative embodiment, neurotensin analogs that are hexapeptides designated NT(8-13) having a 1,2,3,4-tetrahydroisoquinoline at position 11 are described. Additionally, the neurotensin analog may include an N-methyl-arginine at position 8. Additionally, or in the alternative, the neurotensin analog may include a Lysine (D or L) at position 8 and/or position 9, i.e., at at least one of positions 8 or 9. Additionally, or in the alternative, the neurotensin analog may include a tert-leucine at position 12. Additionally, or in the alternative, the neurotensin analog may include an Ornithine (D or L) at position 9. Additionally, or in the alternative, the neurotensin analog may include a diaminobutyric acid at position 9.

In another embodiment, neurotensin analogs that are pentapeptides designated NT(9-13) having a 1,2,3,4-tetrahydroisoquinoline at position 11 are described. Additionally, or in the alternative, the neurotensin analog may include a diaminobutyric acid at position 9. Additionally, or in the alternative, the neurotensin analog may include a Lysine (D or L) at position 9. Additionally, or in the alternative, the neurotensin analog may include a tert-leucine at position 12.

In another embodiment, neurotensin analogs that are pentapeptides designated NT(9-13) having a D-neo-Tryptophan at position 11 are described. Additionally, or in the alternative, the neurotensin analog may include a diaminobutyric acid at position 9. Additionally, or in the alternative, the neurotensin analog may include a Lysine (D or L) at position 9. Additionally, or in the alternative, the neurotensin analog may include a tert-leucine at position 12.

In another embodiment, neurotensin analogs that are hexapeptides designated NT(8-13) having a D-neo-Tryptophan at position 11 are described. Additionally, the neurotensin analog may include an Ornithine (D or L), a diaminobutyric acid, or a Lysine (D or L) at position 9. Additionally, or in the alternative, the neurotensin analog may include an N-methyl-arginine at position 8. Additionally, or in the alternative, the neurotensin analog may include a Lysine (D or L) at position 8. Additionally, or in the alternative, the neurotensin analog may include a tert-leucine at position 12.

In an alternative embodiment, methods for treating pain using any of the above-described analogs are described. The neurotensin analog is provided and administered to a patient in need of pain management. Administration of the neurotensin analog does not substantially reduce the patient's blood pressure. The dosage range for the neurotensin analog could be about 5 to about 20 mg/kg, alternatively about 7 to about 18 mg/kg, alternatively about 10 to about 15 mg/kg, alternatively about 12 to about 15 mg/kg. Alternatively, the dosage may be about 5 mg, alternatively about 7.5 mg, alternatively about 10 mg, alternatively about 12.5 mg, alternatively about 15 mg, alternatively about 17.5 mg, alternatively about 20 mg.

### BRIEF DESCRIPTION OF THE FIGURES

FIG. 1 depicts the structures of unnatural, i.e., synthetic and/or modified, amino acids that were used to make the NT analogs.

FIG. 2 is a graph of a competition binding between radio-labeled NT and NT analogs at NTS2.

FIG. 3 depicts the K_{d}'s for NT(8-13) and NT(9-13) analogs at human NTS1 vs. human NTS2.

FIG. 4 is a graph showing degradation of NT(8-13) and NT(9-13) peptides in human plasma in vitro.

FIG. 5 is a graph of body temperature lowering effects of neurotensin agonists in mice.

FIG. 6 is a graph of the effect of NT79 (20 mg/kg intraperitoneally) on the tail flick and on the hot plate antinociceptive models in rats.

FIG. 7 is a graph of the effect of NT79 (20 mg/kg intraperitoneally) in the acetic acid-induced writhing test in rats.

FIG. 8 is a graph of the effect of saline, NT69 (2 mg/kg intraperitoneally), and NT79 (20 mg/kg intraperitoneally) on plasma prostaglandin levels in mice 30 min after injection. Blood samples from 3 mice were pooled for each condition.

### DETAILED DESCRIPTION

Because of the evidence from animal and human studies suggesting that NT is an endogenous neuroleptic (Bissette G and Nemeroff C B. “The neurobiology of neurotensin.” In: PSYCHOPHARMACOLOGY: THE FOURTH GENERATION OF PROGRESS (Eds. Kupfer D and Bloom F), pp. 573-83. Raven Press, New York (1995); Wolf, S. S. et al. J NEURAL TRANSM 102: 55-65 (1995); Lahti, R. A. et al. J NEURAL TRANSM 105: 507-16 (1998); and Cusack, B. et al. BRAIN RES856: 48-54 (2000)), Dr. Richelson and colleagues have studied NT and its receptors, with the goal of developing a drug that mimics the effects of this neuropeptide. Such a compound possibly could have antipsychotic effects and represent a novel means of treating psychoses. Since the last 6 amino acids of the parent NT, namely NT(8-13) (Arg⁸,Arg⁹,Pro¹⁰,Tyr¹¹,Leu¹³), are sufficient for biological activity at NTS1, these researchers have focused their efforts on analogs of this hexapeptide and analogs of the pentapeptide NT(9-13). Thus, a large number of NT analogs were synthesized that are mostly based on NT(8-13). (See Cusack, B. et al. J BIOL CHEM 270: 18359-66 (1995); Cusack, B. et al. J BIOL CHEM 271: 15054-59 (1996); and Tyler, B. M. et al. NEUROPHARMACOLOGY 38: 1027-34 (1999))

With the availability of this peptide library and the molecularly cloned hNTS1 and hNTS2, the selectivity of these peptides for these receptors was determined from their affinities derived in radioligand binding studies. Most of the compounds tested showed no selectivity for either receptor. A few compounds, however, were both relatively potent and selective (_{>}30 fold higher affinity) at one or the other receptor.

### Peptide Analogs

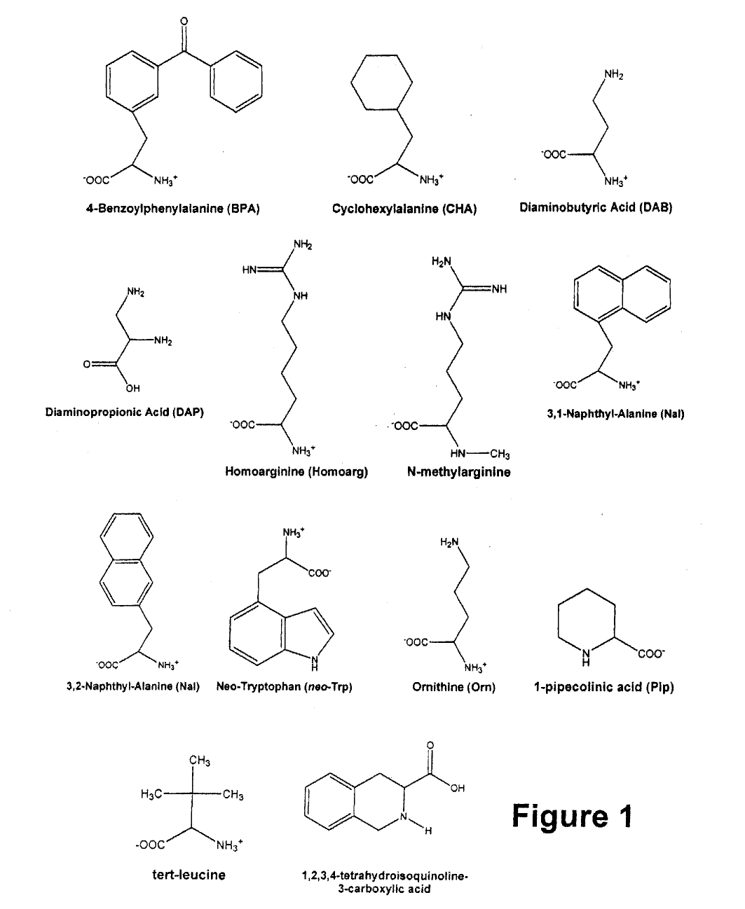

The peptides, which contain unnatural, i.e., synthetic or modified, amino acids, used here and listed in Table 1, were synthesized in the Mayo Peptide Synthesis Facility of the Mayo Proteomics Research Center, formerly known as the Mayo Protein Core Facility (Mayo Clinic, Rochester Minn.), as described in previous publications. (See Morbeck, D. E. et al. “Analysis of hormone-receptor interaction sites using synthetic peptides: receptor binding regions of the alpha-subunit of human choriogonadotropin.” In: Methods: A Companion to Methods in Enzymology, Vol. 5, pp. 191-200. Academic Press, Inc., New York (1993)). The structures of the unnatural amino acids are depicted in FIG. 1. Briefly, all NT peptides were synthesized on automated 433A peptide synthesizers using orthogonal 9-fluorenyl-methoxycarbonyl (Fmoc) protection chemistry with tert-butyl (tBut), tert-butyloxycarbonyl (Boc), 4-methoxy-2,3,6-trimethylbenzenesulphonyl (Mtr) or 2,2,5,7,8-pentamethylchroman-6-sulphonyl (Pmc)-protected side chains. Protocols concerning activation coupling times, amino acid dissolution, coupling solvents and synthesis scale were followed according to the manufacturer's instructions (Applied Biosystems). All peptides were purified by reverse-phase HPLC on silica-bonded C₁₈ columns (Phenomenex or Vydac) in aqueous gradients of 0.1% trifluoroacetic acid (v/v) containing 5% to 80% acetonitrile (v/v) as an organic modifier. The methods of analytical reverse-phase HPLC and ESI-mass spectrometry (ThermoFischer Scientific, MSQ instrument) were used to analyze peptide homogeneity and peptide mass weight, respectively. To prepare the analogs for binding, they were dissolved as 10 mM stock solutions in deionized H₂O, aliquoted in 20-80 μl quantities, and frozen at -30° C. A small number of less hydrophilic compounds were dissolved in DMSO (Sigma Chemical Co., St. Louis, Mo.).

TABLE 1
Amino Acid Sequences of Selected Neurotensin (NT) Analogs

| Polypeptide | 1 | 2 | 3 | 4 | 5 | 6 | 7 | 8 | 9 | 10 | 11 | 12 | 13 |
|---|---|---|---|---|---|---|---|---|---|---|---|---|---|
| NT | p-Glu | L-Leu | L-Tyr | L-Glu | L-Asn | L-Lys | L-Pro | L-Arg | L-Arg | L-Pro | L-Tyr | L-Ile | L-Leu |
| NT02 | | | | | | | | D-Lys | L-Arg | L-Pro | L-Tyr | L-Ile | L-Leu |
| NT03 | | | | | | | | L-Arg | D-Lys | L-Pro | L-Tyr | L-Ile | L-Leu |
| NT04 | | | | | | | | L-Arg | D-Arg | L-Pro | L-Tyr | L-Ile | L-Leu |
| NT06 | | | | | | | | L-Arg | L-Arg | L-Pro | L-Tyr | L-Ile | D-Leu |
| NT07 | | | | | | | | L-Arg | L-Arg | Gly | L-Tyr | L-Ile | L-Leu |
| NT08 | | | | | | | | L-Arg | L-Arg | L-Pro | L-Ala | L-Ile | L-Leu |
| NT09 | | | | | | | | L-Arg | L-Arg | L-Pro | L-Tyr | L-Leu | L-Leu |
| NT10 | | | | | | | | L-Arg | L-Arg | L-Pro | L-Tyr | L-Val | L-Leu |
| NT13 | | | | | | | | D-Arg | L-Arg | L-Pro | L-Tyr | L-Ile | L-Leu |
| NT14 | | | | | | | | D-Arg | D-Arg | L-Pro | L-Tyr | L-Ile | L-Leu |
| NT15 | | | | | | | | D-Arg | L-Lys | L-Pro | L-Tyr | L-Ile | L-Leu |
| NT16 | | | | | | | | L-Lys | D-Arg | L-Pro | L-Tyr | L-Ile | L-Leu |
| NT17 | | | | | | | | L-Lys | L-Arg | L-Pro | L-Tyr | L-Ile | L-Leu |
| NT18 | | | | | | | | L-Arg | L-Lys | L-Pro | L-Tyr | L-Ile | L-Leu |
| NT19 | | | | | | | | L-Lys | L-Lys | L-Pro | L-Tyr | L-Ile | L-Leu |
| NT20 | | | | | | | | D-Lys | D-Lys | L-Pro | L-Tyr | L-Ile | L-Leu |
| NT21 | | | | | | | | L-Orn | L-Arg | L-Pro | L-Tyr | L-Ile | L-Leu |
| NT22 | | | | | | | | D-Orn | L-Arg | L-Pro | L-Tyr | L-Ile | L-Leu |
| NT23 | | | | | | | | L-Arg | L-Orn | L-Pro | L-Tyr | L-Ile | L-Leu |
| NT24 | | | | | | | | L-Arg | D-Orn | L-Pro | L-Tyr | L-Ile | L-Leu |
| NT25 | | | | | | | | L-Orn | L-Orn | L-Pro | L-Tyr | L-Ile | L-Leu |
| NT26 | | | | | | | | L-Orn | D-Orn | L-Pro | L-Tyr | L-Ile | L-Leu |
| NT27 | | | | | | | | D-Orn | L-Orn | L-Pro | L-Tyr | L-Ile | L-Leu |
| NT28 | | | | | | | | D-Orn | D-Orn | L-Pro | L-Tyr | L-Ile | L-Leu |
| NT29 | | | | | | | | DAB | L-Arg | L-Pro | L-Tyr | L-Ile | L-Leu |
| NT30 | | | | | | | | L-Arg | DAB | L-Pro | L-Tyr | L-Ile | L-Leu |
| NT31 | | | | | | | | DAB | DAB | L-Pro | L-Tyr | L-Ile | L-Leu |
| NT32 | | | | | | | | L-Arg | L-Arg | L-Pro | CHA | L-Ile | L-Leu |
| NT33 | | | | | | | | L-Arg | L-Arg | L-Pro | L-3,2- | L-Ile | L-Leu |
| | | | | | | | | | | | Nal | | |
| NT34 | | | | | | | | | L-Orn | L-Pro | L-Tyr | L-Ile | L-Leu |
| NT35 | | | | | | | | | D-Orn | L-Pro | L-Tyr | L-Ile | L-Leu |
| NT36 | | | | | | | | L-Arg | L-Orn | L-Pro | D-Tyr | L-Ile | L-Leu |
| NT37 | | | | | | | | L-Arg | D-Orn | L-Pro | D-Tyr | L-Ile | L-Leu |
| NT38 | | | | | | | | DAP | L-Arg | L-Pro | L-Tyr | L-Ile | L-Leu |
| NT39 | | | | | | | | L-Arg | DAP | L-Pro | L-Tyr | L-Ile | L-Leu |
| NT40 | | | | | | | | DAP | DAP | L-Pro | L-Tyr | L-Ile | L-Leu |
| NT44 | | | | | | | | L-Arg | L- | L-Pro | L-Tyr | L-Ile | L-Leu |
| | | | | | | | | | homoArg | | | | |
| NT45 | | | | | | | | L- | L- | L-Pro | L-Tyr | L-Ile | L-Leu |
| | | | | | | | | homoArg | homoArg | | | | |
| NT46 | | | | | | | | L- | L-Arg | L-Pro | L-Tyr | L-Ile | L-Leu |
| | | | | | | | | homoArg | | | | | |
| NT47 | | | | | | | | L-Arg | L-Arg | L-Pro | L-TIC | L-Ile | L-Leu |
| NT48 | | | | | | | | L-Arg | L-Arg | L-Pro | D-TIC | L-Ile | L-Leu |
| NT49 | | | | | | | | L-Arg | L-Arg | L-Pro | L-3,1- | L-Ile | L-Leu |
| | | | | | | | | | | | Nal | | |
| NT50 | | | | | | | | L-Arg | L-Arg | L-Pro | D-3,1- | L-Ile | L-Leu |
| | | | | | | | | | | | Nal | | |
| NT51 | | | | | | | | L-Arg | L-Arg | L-Pro | D-3,2- | L-Ile | L-Leu |
| | | | | | | | | | | | Nal | | |
| NT52 | | | | | | | | L-Arg | L-Arg | L-Pip | L-Tyr | L-Ile | L-Leu |
| NT54 | p-Glu | L-Leu | L-Tyr | L-Glu | L-Asn | L-Lys | L-Pro | BPA | L-Arg | L-Pro | L-Tyr | L-Ile | L-Leu |
| NT55 | p-Glu | L-Leu | L-Tyr | L-Glu | BPA | L-Lys | L-Pro | L-Arg | L-Arg | L-Pro | L-Tyr | L-Ile | L-Leu |
| NT56 | p-Glu | L-Leu | L-Tyr | L-Glu | L-Asn | L-Lys | L-Pro | L-Arg | BPA | L-Pro | L-Tyr | L-Ile | L-Leu |
| NT59 | | | | | | | | L-Arg | DAB | L-Pro | L-3,1- | L-Ile | L-Leu |
| | | | | | | | | | | | Nal | | |
| NT60 | p-Glu | L-Leu | L-Tyr | L-Glu | L-Asn | L-Lys | L-Pro | L-Arg | L-Orn | L-Pro | L-Tyr | L-Ile | L-Leu |
| NT61 | p-Glu | L-Leu | L-Tyr | L-Glu | L-Asn | L-Lys | L-Pro | L-Arg | D-Orn | L-Pro | L-Tyr | L-Ile | L-Leu |
| NT62 | p-Glu | L-Leu | L-Tyr | L-Glu | L-Asn | L-Lys | L-Pro | L-Arg | L-Arg | L-Pro | L-3,1- | L-Ile | L-Leu |
| | | | | | | | | | | | Nal | | |
| NT64L | | | | | | | | L-Arg | L-Arg | L-Pro | L-neo- | L-Ile | L-Leu |
| | | | | | | | | | | | Trp | | |
| NT65 | | | | | | | | L-Arg | L-Arg | L-Pro | L-neo- | tert-Leu | L-Leu |
| | | | | | | | | | | | Trp | | |
| NT66L | | | | | | | | D-Lys | L-Arg | L-Pro | L-neo- | tert-Leu | L-Leu |
| | | | | | | | | | | | Trp | | |
| NT66T | | | | | | | | D-Lys | L-Arg | L-Pro | L-Trp | tert-Leu | L-Leu |
| NT67L | | | | | | | | D-Lys | L-Arg | L-Pro | L-neo- | L-Ile | L-Leu |
| | | | | | | | | | | | Trp | | |
| NT67T | | | | | | | | D-Lys | L-Arg | L-Pro | L-Trp | L-Ile | L-Leu |
| NT69L | | | | | | | | N- | L-Lys | L-Pro | L-neo- | tert-Leu | L-Leu |
| | | | | | | | | methyl- | | | Trp | | |
| | | | | | | | | Arg | | | | | |
| NT70 | p-Glu | L-Leu | L-iodo- | L-Glu | L-Asn | L-Lys | L-Pro | L-Arg | L-Arg | L-Pro | L-Tyr | L-Ile | L-Leu |
| | | | Tyr | | | | | | | | | | |
| NT71 | | | | | | | | N- | DAB | L-Pro | L-neo- | tert-Leu | L-Leu |
| | | | | | | | | methyl- | | | Trp | | |
| | | | | | | | | Arg | | | | | |
| NT72 | | | | | | | | | D-Lys | L-Pro | L-neo- | tert-Leu | L-Leu |
| | | | | | | | | | | | Trp | | |
| NT73 | | | | | | | | | D-Lys | L-Pro | L-neo- | L-Ile | L-Leu |
| | | | | | | | | | | | Trp | | |
| NT75 | | | | | | | | DAB | L-Arg | L-Pro | L-neo- | L-Ile | L-Leu |
| | | | | | | | | | | | Trp | | |
| NT77 | | | | | | | | L-Arg | D-Orn | L-Pro | L-neo- | tert-Leu | L-Leu |
| | | | | | | | | | | | Trp | | |
| NT77T | | | | | | | | L-Arg | D-Orn | L-Pro | L-Trp | tert-Leu | L-Leu |
| NT78 | | | | | | | | N- | D-Orn | L-Pro | L-neo- | tert-Leu | L-Leu |
| | | | | | | | | methyl- | | | Trp | | |
| | | | | | | | | Arg | | | | | |
| NT78T | | | | | | | | N- | D-Orn | L-Pro | L-Trp | tert-Leu | L-Leu |
| | | | | | | | | methyl- | | | | | |
| | | | | | | | | Arg | | | | | |
| NT79 | | | | | | | | N- | L-Arg | L-Pro | D-3,1- | tert-Leu | L-Leu |
| | | | | | | | | methyl- | | | Nal | | |
| | | | | | | | | Arg | | | | | |
| NT80 | | | | | | | | N- | L-Arg | L-Pro | D-3,1- | L-Ile | L-Leu |
| | | | | | | | | methyl- | | | Nal | | |
| | | | | | | | | Arg | | | | | |

Abbreviations:
BPA = benzoylphenylalanine;
CHA = cyclohexylalanine;
DAB = diaminobutyric acid;
DAP = diaminoproprionic acid;
homoArg = homoarginine;
Orn = ornithine;
Nal = naphthyl-alanine;
NT = neurotensin;
Pip = 1-pipecolinic acid;
neo-Trp = a regio-isomer of the native tryptophan (See Fauq, A. H. et al. “Synthesis of (2S)-2-amino-3-(1H-4-indolyl)propanoic acid, a novel tryptophan analog for structural modification of bioactive peptides.” Tetrahedron: Asymmetry 9: 4127-34 (1998));
TIC = 1,2,3,4-tetrahydroisoquinoline-3-carboxylic acid)

Patent

### Cell Culture

CHO-K1 cells that had been stably transfected separately with the hNTS1 or hNTS2 genes were cultured in 150 mm (500 cm²) Petri plates with 35 ml of Dulbecco's modified Eagle's medium containing 100 μM minimal essential medium nonessential amino acids (Life Technologies, Inc.) supplemented with 5% (v/v) FetalClone II bovine serum product (Hyclone Labs, Logan, Utah). CHO cells (subculture 7-15) were harvested at confluence by aspiration of the medium, followed by a wash with ice-cold 50 mM Tris-HCl buffer, pH=7.4, which was discarded, resuspension in 5-15 ml of Tris-HCl, scraping the cells with a plastic spatula into a centrifuge tube, and collection of cells by centrifugation at 300×g for 5 min at 4° C., in a GPR centrifuge (Beckman Instruments, Fullerton, Calif.). The cellular pellet (in Tris-HCl buffer) was stored at −180° C. until the radioligand binding was performed.

For use in binding assays, crude membrane preparations were prepared by centrifugation of the cellular pellet at 35,600×g for 10 min. The supematant was decanted and discarded, and the cellular pellet was resuspended in 1 ml of Tris-HCl buffer followed by homogenization with a Brinkmann Polytron at setting 5.5 for 15 s. Centrifugation was repeated as above, the supernatant was decanted and discarded, and the cellular pellet was resuspended in 1 ml of Tris-HCl buffer followed by homogenization. Centrifugation was repeated a third time, the supematant was discarded, and the final cellular pellet was suspended in 0.5-2.5 ml of Tris-HCl buffer. Protein concentration of the membrane preparation was estimated by the method of Lowry et al. using bovine serum albumin as a standard. (Lowry O. H. et al. J BIOL CHEM 193: 265-75 (1951)).

### Radioligand Binding Assays

A Biomek 1000 robotic workstation (Beckman Instruments) performed all pipetting steps in the radioligand binding assays as described previously by Cusack et al. J RECEPT RES 13: 123-134, 1993. Competition binding assays with [³H]NT (1 nM), varying concentrations of unlabeled NT, and varying concentrations of peptide analogs were carried out in duplicate in at least three independent experiments with membrane preparations from the appropriate cell lines. Nonspecific binding was determined with 1 μM unlabeled NT in assay tubes with a total volume of 1 ml. Incubation was at 20° C. for 40 min. The assay was routinely terminated by addition of ice-cold 0.9% NaCl (5×1.5 ml), followed by rapid filtration through a GF/B filter strip that had been pretreated with 0.2% or 2% polyethyleneimine. Details of binding assays have been described previously. (See Cusack, B. et al. EUR J PHARMACOL 206: 339-42 (1991)). Data were analyzed using the LIGAND program. (Munson, P. J. and Rodbard, D. ANALYTICAL BIOCHEMISTRY 107: 220-39 (1980)).

### Statistical Analysis

The values presented for equilibrium dissociation constants are expressed as the geometric means+S.E.M. (See Fleming, W. W. et al. J PHARMACOL EXP THER 181: 339-45 (1972) and De Lean, A. MOL PHARMACOL 21: 5-16 (1982)).

### Results

### Radioligand Binding Studies

Results from the radioligand binding studies are listed in Table 2. All the peptides tested had Hill coefficients close to unity (data not shown), indicating binding to a single class of receptors. The most potent compound at both receptors was [L-neo-Trp¹¹]NT(8-13), abbreviated as NT64, with a K_{d}=0.09 nM at hNTS1 and 0.32 nM at hNTS2. Nine analogs had sub-nanomolar K_{d}'s at hNTS1, the data for some of which were reported previously (Table 2). (See Cusack, B. et al. J BIOL CHEM 270: 18359-66 (1995) and Tyler, B. M. et al. NEUROPHARMACOLOGY 38: 1027-34 (1999)). Six analogs had sub-nanomolar K_{d}'s at hNTS2 (Table 2), all but one of which (NT44) also had sub-nanomolar K_{d}'s at hNTS1. Two compounds, [L-Orn⁹,D-Tyr¹¹]NT(8-13) (NT36) and [D-10m⁹,D-Tyr¹¹]NT(8-13) (NT37), had no detectable binding to hNTS1, but had micromolar K_{d}'s at hNTS2. The least potent compounds at hNTS2 were [D-Orn⁹]NT(1-13) (NT61, K_{d}=6.6 μM) and [D-Orn⁹]NT(9-13) (NT35, K_{d}=10 μM).

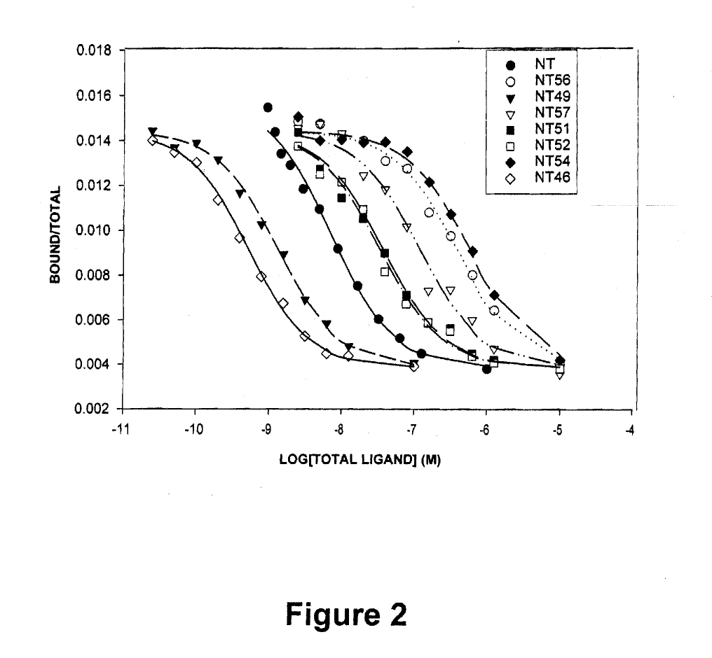

An example of some competition binding curves for compounds at hNTS2, expressed by CHO-K1 cells, is shown in FIG. 2. Assays were performed with membrane preparations, 1 nM [³H]NT, and varying concentrations of compounds as described in the text. Curves were generated using the LIGAND program. (See Munson, P. J. and Rodbard, D. ANALYTICAL BIOCHEMISTRY 107: 220-39 (1980)). Data are the means of duplicate determinations and are representative results from one of at least three independent experiments.

TABLE 2
Radioligand Binding Data for Neurotensin and Analogs at the Human NTS1 and NTS2.

| | | hNTS1 | | hNTS2 | |
|---|---|---|---|---|---|
| Reference | | Geometric | hNTS1 | Geometric | hNTS2 |
| Name | Compound Sequence | Mean ∀ SEM | Selectivity | Mean ∀ SEM | Selectivity |
| NT | Neurotensin | 1.94 ± 0.07 | 3.4 | 6.5 ± 0.1 | 0.3 |
| NT02 | [D-Lys⁸]NT(8-13) | 1.0 ± 0.1† | 4.6 | 4.6 ± 0.5 | 0.2 |
| NT03 | [D-Lys⁹]NT(8-13) | 690 ± 30 | 0.4 | 280 ± 30 | 2.5 |
| NT04 | [D-Arg⁹]NT(8-13) | 158 ± 7 | 0.2 | 24 ± 2 | 6.5 |
| NT06 | [D-Leu¹³]NT(8-13) | 4200 ± 100 | 0.8 | 3300 ± 300 | 1.3 |
| NT07 | [Gly¹⁰]NT(8-13) | 1380 ± 50 | 0.7 | 970 ± 40 | 1.4 |
| NT08 | [Ala¹¹]NT(8-13) | 2500 ± 200 | 0.02 | 58 ± 5 | 43 |
| NT09 | [L-Leu¹²]NT(8-13) | 7.2 ± 0.6 | 0.3 | 2.4 ± 0.3 | 2.9 |
| NT10 | [L-Val¹²]NT(8-13) | 11.3 ± 0.6 | 0.8 | 8.8 ± 0.4 | 1.3 |
| NT13 | [D-Arg⁸]NT(8-13) | 0.50 ± 0.03† | 5.7 | 2.9 ± 0.2 | 0.2 |
| NT14 | [D-Arg⁸,D-Arg⁹]NT(8-13) | 28 ± 3† | 0.7 | 20 ± 2 | 1.4 |
| NT15 | [D-Arg⁸,L-Lys⁹]NT(8-13) | 3.5 ± 0.5‡ | 4.0 | 18 ± 2 | 0.2 |
| NT16 | [L-Lys⁸,D-Arg⁹]NT(8-13) | 33 ± 6† | 1.2 | 39.6 ± 0.6 | 0.8 |
| NT17 | [L-Lys⁸]NT(8-13) | 0.25 ± 0.02† | 4.0 | 1.2 ± 0.2 | 0.2 |
| NT18 | [L-Lys⁹]NT(8-13) | 1.49 ± 0.09‡ | 0.8 | 1.18 ± 0.09 | 1.3 |
| NT19 | [L-Lys⁸,L-Lys⁹]NT(8-13) | 1.4 ± 0.2‡ | 1.7 | 2.4 ± 0.3 | 0.6 |
| NT20 | [D-Lys⁸,D-Lys⁹]NT(8-13) | 185 ± 5† | 4.0 | 730 ± 60 | 0.3 |
| NT21 | [L-Orn⁸]NT(8-13) | 0.41 ± 0.03† | 5.2 | 2.2 ± 0.1 | 0.2 |
| NT22 | [D-Orn⁸]NT(8-13) | 1.9 ± 0.2‡ | 3.2 | 5.9 ± 0.2 | 0.3 |
| NT23 | [L-Orn⁹]NT(8-13) | 0.94 ± 0.06‡ | 1.6 | 1.5 ± 0.1 | 0.6 |
| NT24 | [D-Orn⁹]NT(8-13) | 120 ± 10‡ | 6.6 | 790 ± 20 | 0.2 |
| NT25 | [L-Orn⁸,L-Orn⁹]NT(8-13) | 3.0 ± 0.3‡ | 1.3 | 3.9 ± 0.2 | 0.8 |
| NT26 | [L-Orn⁸,D-Orn⁹]NT(8-13) | 360 ± 40‡ | 3.0 | 1082 ± 6 | 0.3 |
| NT27 | [D-Orn⁸,L-Orn⁹]NT(8-13) | 3.6 ± 0.2† | 6.6 | 24 ± 2 | 0.2 |
| NT28 | [D-Orn⁸,D-Orn⁹]NT(8-13) | 600 ± 20† | 3.2 | 1900 ± 100 | 0.3 |
| NT29 | [DAB⁸]NT(8-13) | 1.2 ± 0.1‡ | 5.6 | 6.5 ± 0.3 | 0.2 |
| NT30 | [DAB⁹]NT(8-13) | 0.41 ± 0.05‡ | 2.2 | 0.90 ± 0.04 | 0.5 |
| NT31 | [DAB⁸,DAB⁹]NT(8-13) | 2.1 ± 0.3‡ | 9.1 | 19.5 ± 0.7 | 0.1 |
| NT32 | [CHA¹¹]NT(8-13) | 1000 ± 200 | 0.1 | 99 ± 2 | 10.1 |
| NT33 | [L-3,2-Nal¹¹]NT(8-13) | 89 ± 9 | 0.2 | 18 ± 1 | 5.0 |
| NT34 | [L-Orn⁹]NT(9-13) | 300 ± 50† | 4.0 | 1190 ± 40 | 0.3 |
| NT35 | [D-Orn⁹]NT(9-13) | 550 ± 80 | 19.1 | 10500 ± 200 | 0.1 |
| NT36 | [L-Orn⁹,D-Tyr¹¹]NT(8-13) | n.d.** | — | 1160 ± 20 | — |
| NT37 | [D-Orn⁹,D-Tyr¹¹]NT(8-13) | n.d. | — | 1800 ± 100 | — |
| NT38 | [DAP⁸]NT(8-13) | 5.8 ± 0.7 | 4.3 | 25 ± 1 | 0.2 |
| NT39 | [DAP⁹]NT(8-13) | 8.6 ± 0.8 | 3.0 | 17.0 ± 0.2 | 0.5 |
| NT40 | [DAP⁸,DAP⁹]NT(8-13) | 175 ± 10 | 6.3 | 1100 ± 30 | 0.2 |
| NT44 | [L-Homoarg⁹]NT(8-13) | 1.7 ± 0.1 | 0.6 | 0.96 ± 0.06 | 1.8 |
| NT45 | [L-Homoarg⁸,L-Homoarg⁹]NT(8-13) | 1.4 ± 0.1 | 0.4 | 0.52 ± 0.02 | 2.6 |
| NT46 | [L-Homoarg⁸]NT(8-13) | 0.41 ± 0.05 | 1.1 | 0.45 ± 0.01 | 0.9 |
| NT47*** | [L-TIC¹¹]NT(8-13) | 720 | 0.02 | 14 | 51.4 |
| NT48*** | [D-TIC¹¹]NT(8-13) | 350 | 0.73 | 255 | 1.4 |
| NT49 | [L-3,1-Nal¹¹]NT(8-13) | 6.4 ± 0.5 | 0.2 | 1.28 ± 0.05 | 5.0 |
| NT50 | [D-3,1-Nal¹¹]NT(8-13) | 1800 ± 500 | 0.01 | 17 ± 3 | 104 |
| NT51 | [D-3,2-Nal¹¹]NT(8-13) | 1080 ± 80 | 0.03 | 32.9 ± 0.6 | 32.8 |
| NT52 | [L-Pip¹⁰]NT(8-13) | 33 ± 6 | 1.2 | 38 ± 2 | 0.9 |
| NT54 | [BPA⁸]NT(1-13) | 18.6 ± 0.9 | 35.5 | 660 ± 50 | 0.03 |
| NT55 | [BPA⁵]NT(1-13) | 0.91 ± 0.09 | 6.2 | 5.7 ± 0.3 | 0.2 |
| NT56 | [BPA⁹]NT(1-13) | 72 ± 8 | 4.6 | 330 ± 40 | 0.2 |
| NT59 | [DAB⁹,L-3,1-Nal¹¹]NT(8-13) | 6.8 ± 0.2 | 0.3 | 1.73 ± 0.09 | 3.9 |
| NT60 | [L-Orn⁹]NT(1-13) | 3.2 ± 0.1 | 5.4 | 17 ± 2 | 0.2 |
| NT61 | [D-Orn⁹]NT(1-13) | 1500 ± 100 | 4.4 | 6600 ± 100 | 0.2 |
| NT62 | [L-3,1 Nal¹¹]NT(1-13) | 8.4 ± 0.3 | 1.7 | 14.2 ± 0.5 | 0.6 |
| NT64L | [L-neo-Trp¹¹]NT(8-13) | 0.09 ± 0.01* | 3.4 | 0.32 ± 0.02 | 0.3 |
| NT65 | [neo-Trp¹¹,tert-Leu¹²]NT(8-13) | 1.01 ± 0.05 | 0.5 | 0.52 ± 0.03 | 1.9 |
| NT66L | [D-Lys⁸,L-neo-Trp¹¹,tert-Leu¹²]NT(8-13) | 10.2 ± 0.6\|\| | 0.7 | 7.1 ± 0.8 | 1.4 |
| NT66T | [D-Lys⁸,L-Trp¹¹,tert-Leu¹²]NT(8-13) | 140 ± 19 | 0.1 | 18.1 ± 0.7 | 7.7 |
| NT67L | [D-Lys⁸,L-neo-Trp¹¹]NT(8-13) | 0.59 ± 0.05\|\| | 2.1 | 1.23 ± 0.03 | 0.5 |
| NT67T | [D-Lys⁸,L-Trp¹¹]NT(8-13) | 17 ± 2 | 0.5 | 8.0 ± 0.4 | 2.2 |
| NT69L | [N-methyl-Arg⁸,L-Lys⁹,L-neo-Trp¹¹,tert-Leu¹²]NT(8-13) | 3.1 ± 0.4 | 0.7 | 2.1 ± 0.2 | 1.5 |
| NT70 | [L-iodo-Tyr³]NT(1-13) | 2.52 ± 0.05 | 1.7 | 4.20 ± 0.04 | 0.6 |
| NT71 | [N-methyl-Arg⁸,DAB⁹,L-neo-Trp¹¹,tert-leu¹²]NT(8-13) | 1.71 ± 0.06 | 0.7 | 1.11 ± 0.03 | 1.5 |
| NT72 | [D-Lys⁹,L-neo-Trp¹¹,tert-Leu¹²]NT(9-13) | 34 ± 9 | 41.0 | 1400 ± 100 | 0.02 |
| NT73 | [D-Lys⁹,L-neo-Trp¹¹]NT(9-13) | 30 ± 3 | 5.5 | 162 ± 3 | 0.2 |
| NT75 | [DAB⁹,L-neo-Trp¹¹]NT(9-13) | 73 ± 5 | 2.3 | 169 ± 8 | 0.4 |
| NT77 | [D-Orn⁹,L-neo-Trp¹¹,tert-Leu¹²]NT(8-13) | 1500 ± 100 | 0.3 | 460 ± 70 | 3.3 |
| NT77T | [D-Orn⁹,L-Trp¹¹,tert-Leu¹²]NT(8-13) | 1530 ± 80 | 0.2 | 320 ± 20 | 4.8 |
| NT78 | [N-methyl-Arg⁸,D-Orn⁹,L-neo-Trp¹¹,tert-Leu¹²]NT(8-13) | 1300 ± 400 | 0.3 | 380 ± 40 | 3.4 |
| NT78T | [N-methyl-Arg⁸,D-Orn⁹,L-Trp¹¹,tert-Leu¹²]NT(8-13) | 1400 ± 300 | 0.5 | 660 ± 50 | 2.1 |
| NT79 | [N-methyl-Arg⁸,D-3,1-Nal¹¹,tert-Leu¹²]NT(8-13) | 1800*** | — | 22 ± 3 | 82 |
| NT80*** | [N-methyl-Arg⁸,D-3,1-Nal¹¹]NT(8-13) | 2000 | — | 30 | 67 |

*Published in Tyler, B. M. et al. “In vitro binding and CNS effects of novel neurotensin agonists that cross the blood-brain barrier.” Neuropharmacology 38: 1027-34 (1999);
†published before in Cusack, B. et al. “Pharmacological and biochemical profiles of unique neurotensin 8-13 analogs exhibiting species selectivity, stereoselectivity, and superagonism.” J Biol Chem 270: 18359-66 (1995);
‡reported before, but numbers are now slightly different from previous numbers (See Cusack, B. et al. J Biol Chem 270: 18359-66 (1995)) because we added more values to obtain the mean;
||Published in Tyler et al. 1999, but these numbers are slightly different, because we added more values to obtain the mean.
**no detectable binding at 1 μM.
***data are not sufficient to calculate geometric mean ± S.E.M.
Abbreviations:
BPA = benzoylphenylalanine;
CHA = cyclohexylalanine;
DAB = diaminobutyric acid;
DAP = diaminoproprionic acid;
Homoarg = homoarginine;
Orn = ornithine;
Nal = naphthyl-alanine;
NT = neurotensin;
Pip = 1-pipecolinic acid;
neo-Trp = a regio-isomer of the native tryptophan (See Fauq, A. H. et al. “Synthesis of (2S)-2-amino-3-(1H-4-indolyl)propanoic acid, a novel tryptophan analog for structural modification of bioactive peptides.” Tetrahedron: Asymmetry 9: 4127-34 (1998));
TIC = 1,2,3,4-tetrahydroisoquinoline-3-carboxylic acid)

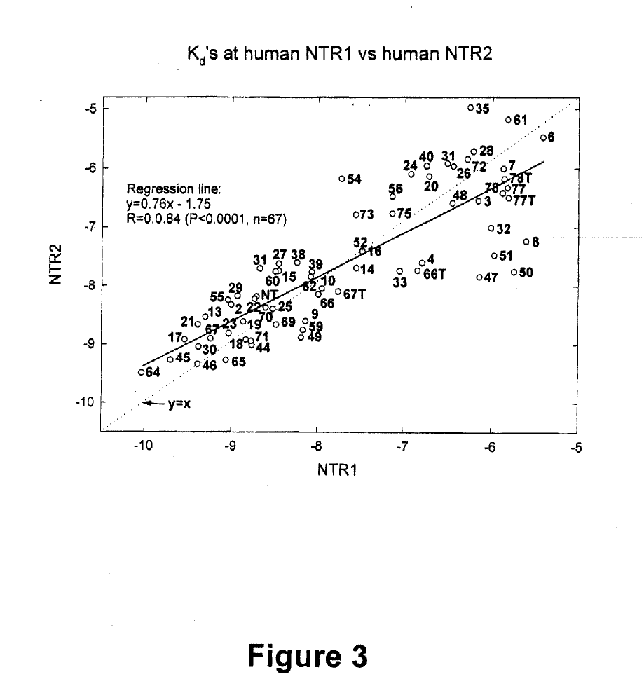

There was a strong correlation between the log K_{d} at hNTS1 and the log K_{d} at hNTS2 (y=0.76x-1.75, R=0.84, P<0.0001) for the peptides (FIG. 3). The relationship between the log K_{d}'s at human NTS1 and human NTS2 is depicted in FIG. 3. The equation for the regression of the log K_{d} at hNTS1 versus the log K_{d} at hNTS2 was y=0.76x-1.75 (R=0.84, P_{<}0.0001). The dashed line is the line of identity. About one-half of the compounds fell at or around the line of identity. There were several compounds, however, that had at least a 10-fold selectivity for one or the other receptor. Thus, three compounds had 19-fold or greater (range 19 to 41 fold) selectivity for hNTS1: [D-Orn⁹]NT(9-13) (NT35, K_{d}=550 nM at hNTS1 and 10500 nM at hNTS2); [BPA¹¹]NT(1-13) (NT54, K_{d}=18.6 nM at hNTS1 and 660 nM at hNTS2); [D-Lys⁹,L-neo-Trp¹¹,tert-Leu¹²]NT(9-13) (NT72, K_{d}=34 nM at hNTS1 and 1400 nM at hNTS2). Five compounds had 10 fold or greater (range 10 to 104 fold) selectivity for hNTS2: [CHA¹¹]NT(8-13) (NT32, K_{d}=1000 nM at hNTS1 and 99 nM at hNTS2); [D-3,2-Nal¹¹]NT(8-13) (NT51, K_{d}=1080 nM at hNTS1 and 32.9 nM at hNTS2); [Ala¹¹]NT(8-13) (NT08, K_{d}=2500 nM at hNTS1 and 58 nM at hNTS2); [L-TIC¹¹]NT(8-13) (NT47, K_{d}=720 nM at hNTS1 and 14 nM at hNTS2); and [D-3,1-Nal¹¹]NT(8-13) (NT50, K_{d}=1800 nM at hNTS1 and 17 nM at hNTS2).

In the present series of peptides, about one-half of the compounds had essentially the same affinities for both hNTS1 and hNTS2 (see FIG. 3, line of identity). Furthermore, there is strong correlation between the log K_{d} at hNTS1 and the log K_{d} at hNTS2 for the peptides. Thus, the binding site for these peptides at the hNTS2 is likely in a region with high homology to the binding site in the hNTS1.

### Receptors

### Compounds Selective for NTS2

In previous publications, Dr. Richelson and colleagues showed the importance of position 11 of NT(8-13) for high-affinity binding to hNTS1. (See Cusack, B. et al. J BIOL CHEM 271: 15054-59 (1996); Pang, Y. P. et al. J BIOL CHEM 271: 15060-68 (1996); and Cusack, B et al. BIOCHEM PHARMACOL 60: 793-801 (2000)). Pi electrons in this position are critical for the cation-pi interactions that contribute to the binding of the ligand to the hNTS1. (See Cusack, B. et al. J BIOL CHEM 271: 15054-59 (1996) and Pang, Y. P. et al. J BIOL CHEM 271: 15060-68 (1996)). It is therefore interesting to note that the most selective compounds at the hNTS2 were compounds with substitutions in position 11: [L-Ala¹¹]NT(8-13), [D-3,1-Nal¹¹]NT(8-13), [L-TIC¹¹]NT(8-13), and [D-3,2-Nal¹¹]NT(8-13). At both receptors, these substitutions reduced the binding affinity, compared to that for NT, for example. The effect, however, was much greater at the hNTS1 than at the hNTS2, leaving very selective and relatively potent compounds at the second subtype.

NT50, [D-3,1-Nal¹¹]NT(8-13), may be the agonist that is selective for NTS2 not only in vitro, but also in vivo based on studies with this compound. After direct injection into the brains of rats, NT50 caused little or no effects on body temperature, but caused behavioral activation (McMahon et al., unpublished observations), results different from those obtained with non-selective agonists. (See Cusack, B. et al. BRAIN RES856: 48-54 (2000) and Tyler-McMahon, B. M. et al. EUR J PHARMACOL 390: 107-11 (2000)).

Of the many NT(8-13) and NT(9-13) peptide analogs that have been synthesized and tested, about 70 have been tested for their affinities at both hNTS1 and hNTS2. Few are selective for either NTS1 or NTS2. Table 3 lists several compounds having selectivity for hNTS2. Based on preliminary in vivo data, NT79 and NT80 have also been found to be selective for NTS2 (not listed in Table 3).

TABLE 3
hNTS2-Selective Compounds

| | | |
|---|---|---|
| hNTS1 | hNTS2 | NTS2 |

| | | |
|---|---|---|
| Compound | K_{d} (nM) | Selectivity |

| | | | |
|---|---|---|---|
| NT08 | 2500 | 58 | 43 |
| NT47 | 720 | 14 | 51 |
| NT50 | 1800 | 17.3 | 104 |
| NT51 | 1080 | 33 | 33 |

The sequences of these compounds are listed in Table 4, along with several other compounds. All compounds, except for NT72, are NT(8-13) analogs. NT72 is an analog of NT(9-13). The four compounds of Table 3 differ from the natural sequence by the single amino acid substitution in position 11. NT(8-13) has L-Tyr in this position.

TABLE 4
Sequences of hNTS2-Selective and hNTS2-Non-Selective Compounds

| | Sequence | | | | | | hNTS2 |
|---|---|---|---|---|---|---|---|
| Compound | 8 | 9 | 10 | 11 | 12 | 13 | Selectivity |
| NT08 | L-Arg | L-Arg | L-Pro | L-Ala | L-Ile | L-Leu | 43 |
| NT47 | L-Arg | L-Arg | L-Pro | L-TIC | L-Ile | L-Leu | 51 |
| NT50 | L-Arg | L-Arg | L-Pro | D-3,1-Nal | L-Ile | L-Leu | 104 |
| NT51 | L-Arg | L-Arg | L-Pro | D-3,2-Nal | L-Ile | L-Leu | 33 |
| NT64 | L-Arg | L-Arg | L-Pro | L-neo-Trp | L-Leu | L-Leu | — |
| NT65 | L-Arg | L-Arg | L-Pro | L-neo-Trp | Tert-Leu | L-Leu | 1.7 |
| NT66 | D-Lys | L-Arg | L-Pro | L-neo-Trp | Tert-Leu | L-Leu | 2 |
| NT67 | D-Lys | L-Arg | L-Pro | L-neo-Trp | L-Ile | L-Leu | — |
| NT69 | N-Me-L-Arg | L-Lys | L-Pro | L-neo-Trp | tert-Leu | L-Leu | 1.5 |
| NT72 | | D-Lys | L-Pro | L-neo-Trp | tert-Leu | L-Leu | — |
| NT77 | L-Arg | D-Orn | L-Pro | L-neo-Trp | tert-Leu | L-Leu | 3.3 |
| NT79 | N-Me-L-Arg | L-Arg | L-Pro | D-3,1-Nal | tert-Leu | L-Leu | 82 |
| NT80 | N-Me-L-Arg | L-Arg | L-Pro | D-3,1-Nal | L-Ile | L-Leu | 67 |

Nal = naphthyl-alanine;
TIC = 1,2,3,4-tetrahydroisoquinoline-3-carboxylic acid;
Orn = ornithine;
“—” indicates higher affinity for hNTS1;
“ND” indicates not yet determined

Dubuc et al. described [3,2-Nal¹¹]NT(8-13) analogs (JMV509 and JMV510) that showed some selectivity for NTS2 receptors (non-human). (See Dubuc, I. et al. J NEUROSCI 19:503-10 (1999)) Their binding assays made use of the molecularly cloned rat NTS1 and the molecularly cloned mouse NTS2. The sequences and binding data are reported in Tables 5A-B below.

TABLE 5A
Sequences of some [3,2-Nal¹¹]NT(8-13) Analogs

| | Sequence | | | | | |
|---|---|---|---|---|---|---|
| Compound | 8 | 9 | 10 | 11 | 12 | 13 |
| NT33 | L-Arg | L-Arg | L-Pro | L-3,2-Nal | L-Ile | L-Leu |
| NT51 | L-Arg | L-Arg | L-Pro | D-3,2-Nal | L-Ile | L-Leu |
| JMV510 | Boc-L-Lys | L-Lys | L-Pro | L-3,2-Nal | L-Ile | L-Leu |
| JMV509 | Boc-L-Lys | L-Lys | L-Pro | D-3,2-Nal | L-Ile | L-Leu |

TABLE 5B
Binding Data of some [3,2-Nal¹¹]NT(8-13) Analogs

| | hNTS1 | hNTS2 | NTS2 |
|---|---|---|---|
| Compound | K_{d} (nM) | | Selectivity |
| NT33 | 89 (human) | 18 (human) | 5 |
| NT51 | 1080 (human) | 33 (human) | 33 |
| JMV510 | 13 (rat) | 215 (mouse) | 0.06 |
| JMV509 | 23000 (rat) | 910 (mouse) | 25 |

There is relatively high homology between the rodent receptors and the human receptors. Specifically, BLAST protein alignment analysis of the deduced amino acid sequences for hNTS1 and rNTS1 indicates 83% identity 89% positives. For hNTS2 and mNTS2, this analysis shows these receptors to have 75% identity and 83% positives. (See Tatusova, T. A. et al. FEMS MICROBIOL LETT174:247-50 (1999))

Despite the relatively high homology, Dr. Richelson and collaborators showed previously and unexpectedly that compounds could bind with much higher affinity to rat NTS1 than to human NTS1. (See Cusack, B. et al. J BIOL CHEM 271:15054-9 (1996)) In fact, one compound that contained L-3,1-Nal in the 11 position bound to the rat receptor 126 fold better than to the human receptor. Additionally, Dr. Richelson and collaborators have never found a compound that bound significantly better to the human receptor than to the rodent receptor. (See Pang, Y. P. et al. J BIOL CHEM 271:15060-8 (1996) and Cusack, B. et al. J BIOL CHEM 270:18359-66 (1995)) Because the binding studies in Dubuc et al. were performed with the molecularly cloned rat NTS1 and the molecularly clone mouse NTS2, it would not have been obvious from their studies that their results would correlate to studies with human molecularly cloned NTS1 and NTS2. Therefore, although in the present case, data are for compounds binding to NTS2, it can be argued strongly that it could not be predicted from the results with murine NTS2 (see Dubuc, I. et al. J NEUROSCI 19:503-10 (1999)) that any of the compounds tested by Dr. Richelson and colleagues would bind with higher affinity to the human receptor than to the rodent receptor.

Table 5B lists the binding data for JMV 509 and NT51, both of which have D-3,2-Nal¹¹, and JMV 510 and NT33, both of which have L-3,2-Nal^{l I}. As described above, previous work found that for all compounds tested, no compound bound significantly better to human NTS1 than to rodent NTS1. Therefore, the results with NT33 and NT51 obtained with human NTS2 could not have been predicted from the results of Dubuc et al. with murine NTS2 and their 3,2-Nal substituted compounds. As seen in Table 5B, the affinities of NT33 and NT51 are much higher at hNTS2 than the affinities of JMV 510 and JMV 509 at mNTS2 (12 and 28 fold higher affinities compared, respectively, to their D- and L-Nal peptides). Although the NTS2 selectivity over NTS1 of JMV 509 (25 fold) is similar to that for NT51 (33 fold), JMV 509 has nearly 1 μM affinity for mNTS2, while NT51 has an affinity of 33 nM, which is nearly 30 fold higher affinity. Furthermore, changing from L- to D-3,2-Nal in our peptides (NT33 compared to NT51) caused less than a 2 fold decrease in affinity at NTS2. In contrast, this change in Dubuc's peptides caused a decrease of more than 4 fold. Finally, changing from L- to D-3,2-Nal in our peptides did not reverse the selectivity of our compounds for hNTS2, as it did for Dubuc et al. That is, both NT33 and NT51 are selective for NTS2 over NTS1, while only JMV 509 has that selectivity.

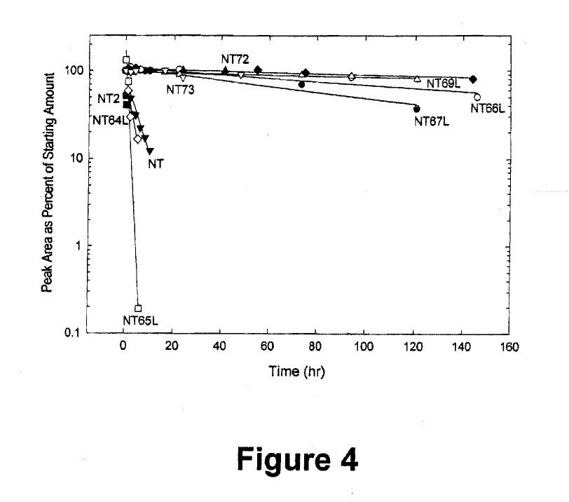

The single property that predicts whether one of the NT(8-13) or NT(9-13) peptides has pharmacological effects in vivo upon injection outside of the brain or spinal cord is stability to degradation by plasma peptidases. As seen in FIG. 4, the results from this simple assay in which peptide was incubated in a test tube with either human (FIG. 4) or rat (data not shown) plasma show that some of the peptides were much more stable than others. All peptides that were stable (half-lives>100 h), such as NT66, NT67, NT69, NT72, and NT73, have either a blocked amino group (N-Methyl-Arg) or a D-amino in the 8 or 9 position (Table 4). Those that lack this feature, such as NT64 and NT65 (Table 4 and FIG. 4) were rapidly degraded.

Virtually all the peptides that had long half-lives in this assay cause their pharmacological effects in brain after administration outside the brain. Likewise, virtually all the short half-life compounds required direct administration into the brain to cause their effects. On this basis, it can be predicted that none of the highly selective compounds at hNTS2 will work by injection outside the brain. Therefore, NT79 and NT80 were designed based on the most selective compound NT50, the sequences for all of which are shown in Table 4. In binding studies with membrane preparations from cells expressing hNTS2, NT79 had a K_{d} of 22 nM (Table 2), close to that found for NT50 (17.3 nM, Table 3), both of which contain D-3,1-Nal” (Table 4). Additionally, in a single experiment with membrane preparations from cells expressing hNTS1, NT79 had a K_{d} of about 1800 nM, giving it a selectivity for hNTS2 of 82 (Table 2). Also, in a single experiment with membrane preparations from cells expressing hNTS1, NT80 had a IQ of about 2000 nM, similar to that for NT79. Furthermore, in two separate experiments with membrane preparations from cells expressing hNTS2, NT80 had a K_{d} of about 30 nM, giving it a selectivity for hNTS2 of 67 (Table 2).

### Antinociceptive Testing

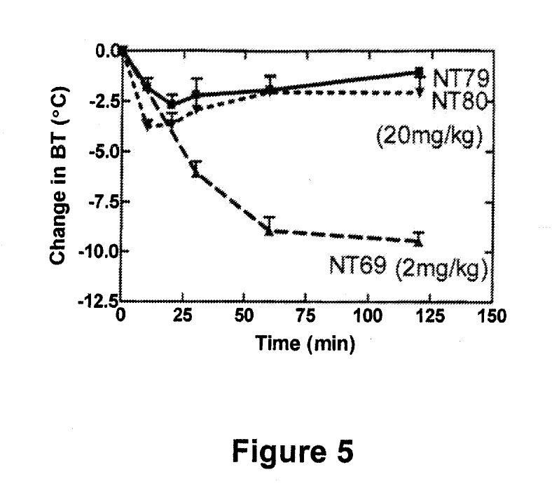

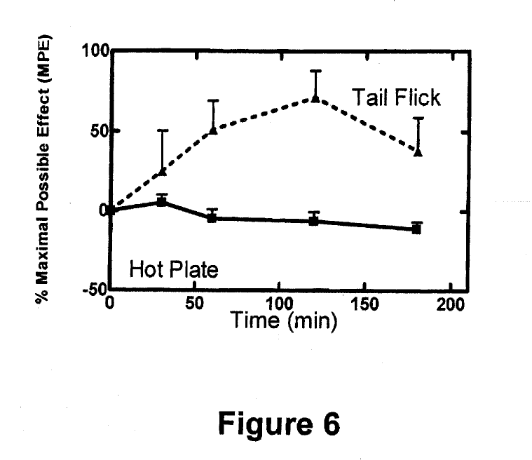

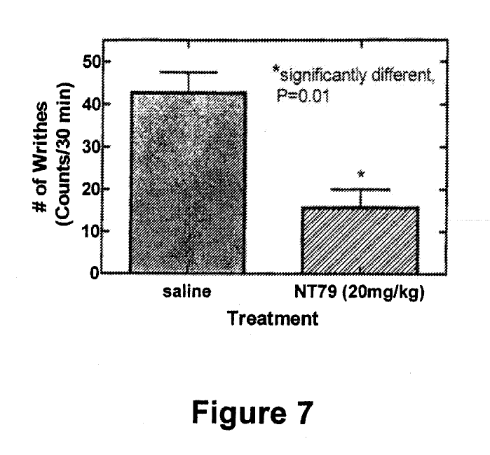

Preliminary data on the pharmacological effects of NT79 and NT80 after intraperitoneal administration to mice (NT79 and NT80, FIG. 5) or to rats (NT79 only, FIGS. 6 and 7) was obtained.

### Body Temperature Lowering

At time “0” baseline readings were made. Afterwards, the mice were injected with a neurotensin analog compound (NT69, NT79, or NT80) and the first reading was taken 30 min after the injection. The thermistor probe was inserted 2 cm into the rectum for the measurement of body temperature.

When injected into the brain, NT causes hypothermia, which indicates a central effect of this peptide on thermal regulation. (See Martin, G. E. et al. PEPTIDES1:333-9 (1980)) NTS1 mediates the hypothermic effects of NT. (See Boules, M. et al. PEPTIDES27:2523-33 (2006)) NT69, an L-neo-Trp NT(8-13) analog is non-selective for the NT-receptor subtypes and has a hypothermic effect. As seen in FIG. 5, administration of NT69 to the mice resulted in a significant change in body temperature (about 10° C. decrease). In contrast, the minimal effects of NT79 and NT80, which were administered at 10 times the dosage of that for NT69, suggest that these compounds have low affinity for NTS1, as we have found in preliminary binding studies (Table 2). Although these results with NT79 and NT80 could also mean that these compounds did not penetrate into brain, this is not consistent with the results of the antinociceptive studies shown in FIGS. 6 and 7. Assuming that these peptides penetrate into brain, these data support the binding data and again suggest that NT79 and NT80 bind weakly to NTS1 and together with the antinociceptive data (FIGS. 6 and 7) have selectivity for NTS2.

### Hot Plate Test

The rats were administered 20 mg/kg of NT79 intraperitoneally. A metal plate (15×20 cm) was heated to 52.5° C. and surrounded by a transparent plastic cage. Baseline testing for the hot plate was measured for each rat immediately prior to the experiment. The latency between the time the rat was placed on the surface and the time it licked either of its hind paws was measured. Failure to respond in a 30 s period resulted in ending the trial and removing the rat from the plate to prevent tissue damage. Hot plate tests were scored as the percentage of Maximal Possible Effect (% MPE) and was calculated according to the following equation:

% MPE=100×(test latency-baseline latency)/(cutoff time {30 s}−baseline latency).

Analgesic compounds will result in higher %MPE.

### Tail Flick Test

The tail flick test also measures changes in nociceptive threshold to thermal stimulus. The rats were administered 20 mg/kg of NT79 intraperitoneally. The rat was placed in a restrainer. Water was heated to 52° C. (52-54° C.). The rat's tail was immersed in the heated water. The latency to flick the tail was recorded. A 10 sec cutoff period was used to prevent tissue damage. Antinociception was expressed as a percentage of the Maximal Possible Effect (MPE) % MPE=100×(test latency-baseline latency)/(cutoff time {10 s}−baseline latency). Analgesic compounds will result in higher %MPE.

### Writhing Test

The writhing test was used to measure changes in the nociceptive threshold to a chemical stimulus. The rats were administered 20 mg/kg of NT79 intraperitoneally. The rats were also injected with 0.5 ml of a 2% (v/v) aqueous solution of acetic acid and placed individually in clear plastic containers for observation.

Behavioral Measure: The number of writhes was counted during a 60 min observation period. A writhe was defined as stretching of the hind limbs accompanied by a contraction of abdominal muscles. Analgesic compounds will result in lower number of writhes.

As seen in FIG. 6, NT79 demonstrated antinociceptive effects in the tail flick assay, but not the hot plate test. Additionally, NT79 had a robust antinociceptive effect in the writhing pain model in rodents (see FIG. 7).

### Prostaglandin Levels

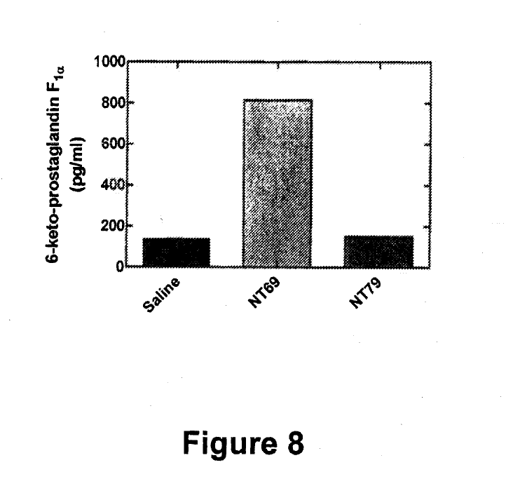

Furthermore, evidence suggests that NTS1 also mediates hypotension. (See Schaeffer, P. et al. EUR J PHARMACOL 323:215-21 (1997)) Therefore, NT79 and NT80 would also be expected to have minimal effects on blood pressure. In this regard, the release of prostacyclins may be related in part to the mechanism whereby NT causes hypotension. (See Schaeffer, P. et al. EUR J PHARMACOL 323:215-21 (1997) and Ertl, G. et al. AM J PHYSIOL 264:H1062-8 (1993)) Consequently, measurement of plasma prostacylin (or its stable metabolite, 6-keto-prostaglandin F_{1α}) may be a surrogate marker for hypotension caused by NT and related compounds. Therefore, in preliminary studies, levels of 6-keto-prostaglandin F_{1α} immunoreactivity were measured after injection of saline, NT69, or NT79 into mice (FIG. 8). Consistent with the literature (See Schaeffer, P. et al. EUR J PHARMACOL 323:215-21 (1997) and Ertl, G. et al. AM J PHYSIOL 264:H1062-8 (1993)) and because it causes hypotension, NT69 markedly elevated plasma levels of prostaglandin. On the other hand, as seen in FIG. 8, NT79 had no effect on these levels, compared to the saline-injected animal. These data suggest that NT79 did not cause hypotension.

### Additional Compounds

The peptides listed in Tables 6A-D were designed to provide hNTS2-selectivity and stability to degradation by peptidases. Rules for this latter feature have come from extensive studies on NT(8-13) and NT(9-13) peptide analogs (e.g., FIG. 4). Additionally, it has been observed in binding studies with hNTS1 and hNTS2 with these analogs that tert-Leu reduces affinity of peptides at both receptors, but more so at hNTS1 than at hNTS2. Radioligand binding studies on hNTS1 and hNTS2 are performed on all the compounds using the protocol described previously. Additional pharmacological studies, including antinociceptive tests, are performed on those analogs showing selectivity for hNTS2.

Peptides (about 30 mg of peptide (>95%) purity) are synthesized using Fmoc chemistry with tBut, Boc, Mtr, or Pmc protected side chains, on an automated 433A peptide synthesizer (Perkin-Elmer/Applied Biosystems, Foster City, Calif.) or by simultaneous methods on an APEX 396 multiple peptide synthesizer (AAPPTEC). Protocols for activation, coupling times, amino acid dissolution, coupling solvents, and synthesis scales at either 40 or 100 μmol are followed according to the manufacturer's programs. The NT peptides are purified by reverse-phase HPLC using a semi-preparative C₁₈ column (2.2 cm×25 cm, Phenomenex, Hesperia, Calif.) in aqueous solutions of 0.1% trifluoroacetic acid and an aqueous gradient of 10%-60% acetonitrile in 0.1% trifluoroacetic acid. A combination of analytical reverse-phase HPLC and electrospray ionization (ESI) mass spectrometry (MSQ, ThermoFischer Scientific) was used to analyze peptide homogeniety and to confirm peptide molecular weight, respectively.

TABLE 6A
NT(8-13) and NT(9-13) D-3,1-Napthylalanine¹¹ Analogs

| Com- | Sequence | | | | | |
|---|---|---|---|---|---|---|
| pound | 8 | 9 | 10 | 11 | 12 | 13 |
| 1 | | DAB | L-Pro | D-3,1-Nal | L-Ile | L-Leu |
| 2 | | DAB | L-Pro | D-3,1-Nal | tert-Leu | L-Leu |
| 3 | | D-Lys | L-Pro | D-3,1-Nal | L-Ile | L-Leu |
| 4 | | D-Lys | L-Pro | D-3,1-Nal | tert-Leu | L-Leu |
| 5 | D-Lys | L-Arg | L-Pro | D-3,1-Nal | L-Ile | L-Leu |
| 6 | D-Lys | L-Arg | L-Pro | D-3,1-Nal | tert-Leu | L-Leu |
| 7 | L-Arg | D-Orn | L-Pro | D-3,1-Nal | L-Ile | L-Leu |
| 8 | L-Arg | D-Orn | L-Pro | D-3,1-Nal | tert-Leu | L-Leu |
| 9 | N-methyl-Arg | DAB | L-Pro | D-3,1-Nal | L-Ile | L-Leu |
| 10 | N-methyl-Arg | DAB | L-Pro | D-3,1-Nal | tert-Leu | L-Leu |
| 11 | N-methyl-Arg | D-Orn | L-Pro | D-3,1-Nal | L-Ile | L-Leu |
| 12 | N-methyl-Arg | D-Orn | L-Pro | D-3,1-Nal | tert-Leu | L-Leu |
| 13 | N-methyl-Arg | L-Lys | L-Pro | D-3,1-Nal | L-Ile | L-Leu |
| 14 | N-methyl-Arg | L-Lys | L-Pro | D-3,1-Nal | tert-Leu | L-Leu |

TABLE 6B
NT(8-13) and NT(9-13)
L-1,2,3,4-Tetrahydroisoquinoline-3-Carboxylic Acid¹¹ Analogs

| Com- | Sequence | | | | | |
|---|---|---|---|---|---|---|
| pound | 8 | 9 | 10 | 11 | 12 | 13 |
| 15 | | DAB | L-Pro | L-TIC | L-Ile | L-Leu |
| 16 | | DAB | L-Pro | L-TIC | tert-Leu | L-Leu |
| 17 | | D-Lys | L-Pro | L-TIC | L-Ile | L-Leu |
| 18 | | D-Lys | L-Pro | L-TIC | tert-Leu | L-Leu |
| 19 | D-Lys | L-Arg | L-Pro | L-TIC | L-Ile | L-Leu |
| 20 | D-Lys | L-Arg | L-Pro | L-TIC | tert-Leu | L-Leu |
| 21 | L-Arg | D-Orn | L-Pro | L-TIC | L-Ile | L-Leu |
| 22 | L-Arg | D-Orn | L-Pro | L-TIC | tert-Leu | L-Leu |
| 23 | N-methyl-Arg | DAB | L-Pro | L-TIC | L-Ile | L-Leu |
| 24 | N-methyl-Arg | DAB | L-Pro | L-TIC | tert-Leu | L-Leu |
| 25 | N-methyl-Arg | D-Orn | L-Pro | L-TIC | L-Ile | L-Leu |
| 26 | N-methyl-Arg | D-Orn | L-Pro | L-TIC | tert-Leu | L-Leu |
| 27 | N-methyl-Arg | L-Lys | L-Pro | L-TIC | L-Ile | L-Leu |
| 28 | N-methyl-Arg | L-Lys | L-Pro | L-TIC | tert-Leu | L-Leu |

DAB = diaminobutyric acid;
tert-Leu = tertiary leucine;
D-Orn = D-Ornithine

TABLE 6C
NT(8-13) and NT(9-13) L-Alanine¹¹ Analogs

| | Sequence | | | | | |
|---|---|---|---|---|---|---|
| Compound | 8 | 9 | 10 | 11 | 12 | 13 |
| 29 | | DAB | L-Pro | L-Ala | L-Ile | L-Leu |
| 30 | | DAB | L-Pro | L-Ala | tert-Leu | L-Leu |
| 31 | | D-Lys | L-Pro | L-Ala | L-Ile | L-Leu |
| 32 | | D-Lys | L-Pro | L-Ala | tert-Leu | L-Leu |
| 33 | D-Lys | L-Arg | L-Pro | L-Ala | L-Ile | L-Leu |
| 34 | D-Lys | L-Arg | L-Pro | L-Ala | tert-Leu | L-Leu |
| 35 | L-Arg | D-Orn | L-Pro | L-Ala | L-Ile | L-Leu |
| 36 | L-Arg | D-Orn | L-Pro | L-Ala | tert-Leu | L-Leu |
| 37 | N-methyl-Arg | DAB | L-Pro | L-Ala | L-Ile | L-Leu |
| 38 | N-methyl-Arg | DAB | L-Pro | L-Ala | tert-Leu | L-Leu |
| 39 | N-methyl-Arg | D-Orn | L-Pro | L-Ala | L-Ile | L-Leu |
| 40 | N-methyl-Arg | D-Orn | L-Pro | L-Ala | tert-Leu | L-Leu |
| 41 | N-methyl-Arg | L-Lys | L-Pro | L-Ala | L-Ile | L-Leu |
| 42 | N-methyl-Arg | L-Lys | L-Pro | L-Ala | tert-Leu | L-Leu |

TABLE 6D
NT(8-13) and NT(9-13) D-neo-Trp¹¹ Analogs

| Com- | Sequence | | | | | |
|---|---|---|---|---|---|---|
| pound | 8 | 9 | 10 | 11 | 12 | 13 |
| 43 | | DAB | L-Pro | D-neo-Trp | L-Ile | L-Leu |
| 44 | | DAB | L-Pro | D-neo-Trp | tert-Leu | L-Leu |
| 45 | | D-Lys | L-Pro | D-neo-Trp | L-Ile | L-Leu |
| 46 | | D-Lys | L-Pro | D-neo-Trp | tert-Leu | L-Leu |
| 47 | D-Lys | L-Arg | L-Pro | D-neo-Trp | L-Ile | L-Leu |
| 48 | D-Lys | L-Arg | L-Pro | D-neo-Trp | tert-Leu | L-Leu |
| 49 | L-Arg | D-Orn | L-Pro | D-neo-Trp | L-Ile | L-Leu |
| 50 | L-Arg | D-Orn | L-Pro | D-neo-Trp | tert-Leu | L-Leu |
| 51 | N-methyl-Arg | DAB | L-Pro | D-neo-Trp | L-Ile | L-Leu |
| 52 | N-methyl-Arg | DAB | L-Pro | D-neo-Trp | tert-Leu | L-Leu |
| 53 | N-methyl-Arg | D-Orn | L-Pro | D-neo-Trp | L-Ile | L-Leu |
| 54 | N-methyl-Arg | D-Orn | L-Pro | D-neo-Trp | tert-Leu | L-Leu |
| 55 | N-methyl-Arg | L-Lys | L-Pro | D-neo-Trp | L-Ile | L-Leu |
| 56 | N-methyl-Arg | L-Lys | L-Pro | D-neo-Trp | tert-Leu | L-Leu |

DAB = diaminobutyric acid;
tert-Leu = tertiary leucine;
D-Orn = D-Ornithine

Radioligand binding studies are performed as detailed above to determine the equilibrium dissociation constants (K_{d}) for the additional compounds for NTS1 and NTS2 to determine which compounds have selectivity for NTS2. Additionally, stability tests with plasma peptidases, prostaglandin level tests, and antinociceptive tests are performed as described above.

Although the foregoing invention has, for the purposes of clarity and understanding, been described in some detail by way of illustration and example, it will be obvious that certain changes and modifications may be practiced which will still fall within the scope of the appended claims.

What is claimed:

1. A neurotensin analog comprising a hexapeptide designated NT(8-13) having a D-3,1-naphthyl-alanine at position 11.

2. The neurotensin analog of claim 1, further comprising an N-methyl arginine at position 8.

3. The neurotensin analog of claim 2, further comprising a tert-leucine at position 12.

4. The neurotensin analog of claim 2, further comprising a diaminobutyric acid at position 9.

5. The neurotensin analog of claim 1, further comprising a D-Lysine at position 8.

6. The neurotensin analog of claim 5, further comprising a tert-leucine at position 12.

7. The neurotensin analog of claim 1, further comprising a tert-leucine at position 12.

8. The neurotensin analog of claim 1, further comprising a D-Ornithine at position 9.

9. The neurotensin analog of claim 1, further comprising an L-Lysine at position 9.

10. A neurotensin analog comprising a pentapeptide designated NT(9-13) having a 3,1-naphthyl-alanine at position 11.

11. The neurotensin analog of claim 10, wherein the neurotensin analog has a D-3,1-naphthyl-alanine at position 11.

12. The neurotensin analog of claim 10, further comprising a diaminobutyric acid at position 9.

13. The neurotensin analog of claim 10, further comprising a D-Lysine at position 9.

14. The neurotensin analog of claim 10, further comprising a tert-leucine at position 12.

15. A neurotensin analog comprising a hexapeptide designated NT(8-13) having a D-3,2-naphthyl-alanine at position 11 with the proviso that positions 8 and 9 are not Lysine.
